(19) United States
(12) Patent Application Publication
(10) Pub. No.: US 2023/0350983 A1
(43) Pub. Date: Nov. 2, 2023

(54) Control center and licensing management system for an intraoral sensor

(71) Applicants: Todd C. Miller, San Jose, CA (US); Karsten I. Christiansen, San Jose, CA (US)

(72) Inventors: Todd C. Miller, San Jose, CA (US); Karsten I. Christiansen, San Jose, CA (US)

(21) Appl. No.: 18/207,036

(22) Filed: Jul. 24, 2023

Related U.S. Application Data
(63) Continuation-in-part of application No. 16/991,380, filed on Aug. 12, 2020.

(51) Int. Cl. G06F 21/10

(52) U.S. Cl. CPC G06F 21/107

(57) ABSTRACT

A sensor licensing system for an intraoral sensor includes an on/off mechanism, a driver, and a sensor. The on/off mechanism is coupled to the intraoral sensor and has an encrypted licensing code so that the on/off mechanism turns on and off the intraoral sensor. The driver processes the encrypted license code stored in the sensor memory and determines if license code is expired based on the computer clock, exposure counter or other desired limits. The sensor periodically checks an online server for an updated encrypted license code and stores new license code in sensor memory. The updated license code is created and stored on the server when a new periodic subscription payment is made. The sensor periodically checks and downloads any updated license code. Failure to make a payment will result in no encrypted license code update being created and eventual expiration of the old, encrypted license code.

### BACKGROUND OF THE INVENTION

### Field of the Invention

The present invention relates to a licensing management system for an intraoral sensor and more particularly to management of licenses, licensing data, tamper protection, x-ray dose optimization, exposure monitoring and other status/support information.

### Description of the Prior Art

In order to x-ray images of teeth and a jawbone by using an x-ray imaging method in the related art, there is used a method for continuously photographing the teeth and the jawbone over and over again, including: inserting a sensor or film into the oral cavity, positioning an intraoral x-ray unit for x-ray imaging outside the patient, irradiating x-ray, and changing the position of the sensor again.

In the former method, a hard square-shaped sensor is inserted into the oral cavity of a patient so that the patient feels a foreign object in the oral cavity. It is impossible to exactly maintain the position of the sensor during imaging and the sensor is not manufactured to fit the oral structure of each patient, and a case where an actually desired image may not be obtained with one x-ray exposure occurs and a process of again positioning the sensor and capturing an image is repeated, thereby making the patient feel uncomfortable and resulting in irradiation of unnecessary radiation.

U.S. Pat. No. 8,119,990 teaches a system for automatic detection of x-rays at an x-ray sensor. A source emits x-ray radiation towards an x-ray sensor, and the x-ray sensor automatically detects the x-ray radiation. The x-ray sensor automatically detects x-ray radiation by evaluating a time series and determining that a voltage threshold is crossed a certain amount of time earlier than the average time it takes the voltage threshold to be crossed from dark current and other noise. X-rays have been used in dentistry to image teeth and parts of the mouth for many years. In general, the process involves generating x-rays outside the patient’s oral cavity and directing the x-rays at an image receptor located in the patient’s mouth. The x-rays are attenuated differently by various parts of the patient’s dental structures (e.g., bone versus tissue) and this difference in attenuation is used to create an image, such as on film or by using an electronic image sensor. In most cases, the x-ray source is triggered manually by the operator. In other words, the capturing of an image is initiated by a technician or other person by activating a switch. In the case of film-based systems, the image is captured as soon as the film is exposed to x-ray radiation. So, there is no need to “activate” the film. Once the x-ray source is activated and the x-rays reach the film, an image is captured.

In electronic systems, the image which is captured depends on at least two factors: activation of the x-ray source and “activation” of the sensor. What constitutes “activation” of the sensor can vary based upon the type of sensor used, but in most cases “activation” occurs when a command is provided to the sensor to either store or output its current image data (referred to herein as “image capture”). So, in some systems, there is an electrical link between the x-ray source and the sensor such that when the x-ray source is activated, a command is sent (simultaneously or simultaneously) to the sensor to perform an image capture. It is possible to generate a burst of x-ray radiation and be assured that an image will be captured by the sensor during the brief period of x-ray exposure. Another challenge associated with automatic triggering systems relates to the alignment between the x-ray source and the sensor. In many instances, even with the use of a positioning system or mechanism, x-ray sensors (particularly those placed in the mouth (i.e., an intra-oral sensor)) are often misaligned. Only a portion of the x-ray sensor is exposed to radiation. In many instances, this partial exposure is not sufficient to cause a simple threshold-based trigger to initiate image capture. A misalignment may not be recognized until the x-ray technician attempts to review images that he or she believes to have been created only to discover that no such images have been created. The technician may then try to realign the x-ray source and sensor and reinitiate the imaging process. It may take several attempts to capture a usable image and each attempt exposes the patient to additional doses of x-ray radiation. As is well-known, high doses of x-ray radiation can have severe adverse effects on an individual’s health. So, unnecessary exposure to x-rays should be avoided.

U.S. Pat. Application Publication No. 2002/0107809 teaches a license management system for managing licensing data that may be applied to any property, product and/or service licensing model. The licensing management system includes client and server managed security features to control or otherwise monitor and/or restrict the use and re-distribution of licensed subject matter.

Many commercial transactions are based on the licensing of property, products or services which may involve a limitation of the scope and/or duration of their use. Accordingly, licensing contracts may operate to restrict or otherwise limit the user’s ability to assign, redistribute, resale or otherwise alter the intended beneficiary of the license, while other restrictions may be directed to how, when, where and for how long the use may occur. Both parties generally derive an economic benefit from structuring a transaction in such a fashion: (1) the licensor retains ownership interest in the subject of the license and control over who may make, use or sell the same, and (2) the licensee enjoys the benefit of using the property, product or service at a reduced cost as compared to the licensee having to (a) acquire the subject of the license by freehold, or (b) develop the property, product or service at the expense of other capital resources.

With respect to licensor transactions, the growth of the global market has been a factor in licensing being refined and developed to better serve both economic and strategic business goals. Where licensor ‘A’ grants a license to licensee ‘B’, such a license may be granted in consideration of a license granted back from licensee ‘B’ to original licensor ‘A’ (e.g., a cross license). In these circumstances, both party ‘A’ and party ‘B’ will be deemed to have determined that the granting of a cross license strengthens or otherwise prospectively improves their relative positions in the marketplace. Licenses that have been granted, received, and/or traded may be accumulated into a licensing portfolio having tangible economic value and may be leveraged or borrowed to fund capital resource development.

With respect to licensee transactions, individuals encounter a variety of circumstances in their daily lives that involve the licensing of goods and services, such as, for a house or apartment, renting a hotel room, purchasing a ticket to an amusement park, watching a film at a movie theatre, renting or leasing a car, purchasing a parking pass, obtaining permission from the state to operate a motor vehicle or to place a particular motor vehicle in service, purchasing a membership at a discount warehouse store, flying on a commercial airline, using a passport or obtaining a visa to enter a foreign country, making a telephone call with a calling card, using computer software applications, casting a vote at a shareholder’s annual meeting, or joining a country club.

With the growth of licensing business models, problems involving the efficient distribution, authorized conveyance, tracking, and management of licenses, both by licensees and licensors, have grown as well. In the commercial software industry software application products have been sold on a purchase basis with license agreements for limited use of the software. Sales representatives often market the software to prospective end-users and, upon purchase in a conventional fashion, the software is then provided to the user on diskettes or other media along with user manuals. Many software applications have been sold primarily on a long-term or permanent license basis with support services being provided under long-term, fixed-price contracts.

From an end-user’s perspective, software acquisition under a conventional purchase-based license agreement can be expensive. Specifically, once an end-user initially invests in a conventional software purchase, the purchase-based acquisition of additional software titles from other vendors may not be feasible. The vendor may charge the user for application upgrades and continuing product support. In this regard, many end-users may become overly dependent on a particular vendor and/or application product. Under such circumstances, the end-user may not have the flexibility to manage costs efficiently. Even though the software may only be required several months out of a year or few times in the user’s development cycle, the user typically still obtains a long-term license to use the software under the terms of a conventional purchase-based license agreement. This can be disadvantageous for end-users, particularly considering the limited shelf-life of most software titles. Because resources are already allocated, end-users may experience constraints on their ability to acquire or convert to superior software tools and services as they may become available.

From a software application vendor’s perspective, a large portion of revenue is spent on sales, marketing and user support through direct sales and the use of VAR (value added reseller) channels. Internet access and the proliferation of high-speed connections (i.e., T1, cable and DSL) have made the electronic distribution of software application products more feasible. As the popularity and accessibility of the Internet has grown, vendors have increasingly looked to the Internet as an effective medium for reducing sales and marketing costs. As a result, some vendors have expanded to support electronic purchase and delivery of software applications over the Internet, but under the conventional purchase-based license agreement model discussed immediately above. The prior art has not solved the problem of providing a comprehensive method to manage, track and customize, inter alia, software application licenses. In addition to cost and efficiency concerns, vendors often are confronted with the issue of software piracy and other unlicensed, unauthorized, or illegal use. As a result, vendors have implemented certain security features within software products to protect the application from unlicensed use. The vendor may find that expensive additional resources are required to support the licensing security features in addition to support for the application itself in many instances, the support for an application may include live telephone support. As many as 50% of the technical support calls that a vendor receives may involve licensing issues. This can prove to be a burden on the vendor’s available development resources. Having an extensible licensing management system to use for the electronic distribution of application products would improve the ability of software vendors to reallocate resources more efficiently to application development. The electronic distribution of software applications also poses a security risk for many vendors. Conventionally, where an encryption method may be employed to protect the software code, protection after decryption of the software may be minimal or non-existent. Accordingly, once the software has been delivered to the end-user’s platform, it may be difficult for the vendor to protect against tampering and software piracy. The electronic security solutions implemented by vendors are not necessarily safe for the user. The user may be required to maintain a data connection with the Application Service Provider (ASP) while using the distributed software. An ASP working environment may also require the user to upload potentially sensitive data to the vendor site, thereby introducing security issues in those circumstances where the user may be excluded from running the distributed application if access to the vendor site is occupied by other users.

Software application vendors are faced with several challenges when trying to establish and manage product licensing for their products, such as managing order entry, tracking product use, offering multiple licensing options, integrating license management into product installation, ensuring licensing information and product source are secure, managing distributed support across a network, customizing the licensing process, giving end-user’s control over how their licenses are dynamically distributed and employed, and controlling the cost and complexity of license management.

There is a need to address these, and similar deficiencies associated with the effective and efficient management of licensing in a wide variety of licensee and licensor market environments. With respect to the software application industry, a need exists for a turnkey electronic method for obtaining licenses and distributing software applications on an as needed basis. A need also exists for a network-based system that allows software user’s online access to a variety of application tools that may be used on a trial basis to determine product usefulness and then purchased in a secure, convenient fashion for as long as the application product is needed or on a subscription basis. A need also exists for a secure method for vendor distribution of software and for maintaining security on the end-user’s platform. A need also exists for dynamic user-level management of the distribution and use of software application licenses.

Hardware application vendors are faced with several challenges when trying to establish and manage product licensing for their products, such as: managing order entry, tracking product use, offering multiple licensing options, integrating license management into product installation, ensuring licensing information and product source are secure, managing distributed support across a network, customizing the licensing process, giving end-user’s control over how their licenses are dynamically distributed and employed and controlling the cost and complexity of license management. There is a need to address these, and similar deficiencies associated with the effective and efficient management of licensing in a wide variety of licensee and licensor market environments. With respect to the hardware device industry, a need exists for a turnkey electronic method for obtaining licenses on an as needed basis. A need also exists for a network-based system that allows user’s online access to a variety of application tools that may be used on a trial basis to determine product usefulness and then purchased in a secure, convenient fashion for as long as the application product is needed or on a subscription basis.

Software or information piracy is the activity of using or making copies of software or information without the authorization of the creator or legitimate owner of that software or information. Piracy is most prevalent in the computer software application industry where people frequently make unlicensed illegal copies of a software application. The application may be copied for personal use or for re-production and commercial profit. Other types of piracy include acts of copying information such as musical recordings or an electronically readable version of documentation or an electronic book. In all cases, piracy costs billions of dollars of lost profits to business annually.

The software and information technology industries have responded to the threat of piracy using locking schemes. Locking schemes can include software locking mechanisms, licenses and specialized hardware devices which prevent unauthorized use of software, information, or an entire electronic device. These schemes seek to prevent adversaries from being able to freely copy software.

There are many types of software locking mechanisms. A manufacturer can encrypt portions of a software program with a unique key. A customer who purchases the software is given the key which allows decryption and execution of the software. Such a software protection mechanism is a “Certificate of Authenticity” supplied with the purchase of software programs such as Microsoft Windows 98, manufactured by the Microsoft Corporation of Redmond, Wash. Microsoft and Windows98 are trademarks of the Microsoft Corporation. The Certificate of Authenticity indicates a unique product number. During installation of the software, the product number is requested by the software application and must be entered correctly by the user. If the product number entered matches a number expected by the application, the copy of the application is assumed to be legitimate and is allowed to be installed and executed as normal. If the number entered is incorrect, the software will not be installed properly.

Hardware piracy protection schemes attach a device to the processor, typically through a communications port. These types of hardware devices are often called “dongles.” U.S. Pat. No. 3,996,449 teaches a hardware protection scheme which is a method for determining if a program or a portion of a program is valid when running on a computer. A hash function is applied to a user’s identification code or key along with the text of the program itself in a special tamper-proof hardware checking device. The checking device compares a resulting value from the hash function with a verifier value to see if the program text is correct. If the text is correct the program is allowed to execute on the device.

Another hardware related approach assigns a unique identifier to each processor that can execute programs. Software programs are then encoded with the identity of a designated processor identifier to which that program is assigned or authorized to execute. No other processor identifications are provided for the software and the software will not run on other processors. Obviously, such systems can provide usage limitations when attempting to execute software on a processor with which that software is not specifically associated. The number assignment mechanism may be supervised using an authorization network which can associate a piece of software with a specific processor identification number.

Aside from the electronic hardware and computer software application and data protection mechanisms noted above, little has been done to thwart the piracy of other types of encoded information that is accessed by electronic devices, such as musical recordings.

U.S. Pat. Application Publication No. 2002/0107809 also teaches n a method for managing licensing data which includes the steps of providing a host system having a processor for processing digital data and providing a client system having a processor for processing digital data and communicably connected to the host system. The method also includes the steps of providing a license management host application running on the host system with the host application having access to a licensing database and providing a license management client application running on the client system with client application having access to a client license datastore. The method further includes the steps of providing a user interface configured to accept licensing orders with the user interface providing a user with access for ordering a license, said user interface requesting the issuance of a license, issuing a license from the licensing database, using the management host application, in response to a user interface request to add a license to the client license datastore, monitoring usage of a license so issued using the management host application, compiling and displaying at least a plurality of licenses stored in the client license datastore using the license management client application and communicating with the host application thereby using the license management client application to modify terms corresponding to at least one license stored in the client license datastore.

U.S. Pat. No. 9,259,197 teaches an intraoral x-ray sensor with embedded standard computer interface. The intraoral x-ray sensor includes a data transfer cable. The cable is a quad-twisted USB cable and includes two data lines, a ground line, and fillers twisted within a metallic sheath. The cable is symmetrically organized about a centerline. The symmetric cable has an improved life due to the ability to withstand mechanical stress (e.g., rotational stress). The intraoral x-ray sensor includes a processor and a housing with an inner metallization layer. The sheath is coupled to the inner metallization layer to transfer heat generated by the processor from the inner metallization layer to the sheath.

U.S. Pat. No. 9,492,129 teaches automatic triggering of an intraoral x-ray sensor which is used in a dental x-ray imaging system. The intraoral sensor has an array of pixels. The array of pixels has a plurality of lines of pixels, and each of the pixels generates an electrical signal correlated to x-ray radiation that impinges that pixel. An electronic control unit is connected to the intraoral sensor to receive electric signals from the array of pixels. The electronic control unit destructively reads pixel clusters in one or more of the plurality of lines of pixels. The electronic control unit is configured to generate a dose-correlated signal based on the signals from each of the pixel clusters in each of the one or more lines of pixels and initiate capture of an image generated with information from each of the pixels in the array of pixels, when the combined signal exceeds a predetermined threshold.

U.S. Pat. No. 9,050,040 teaches an x-ray imaging system for capturing an x-ray image of teeth and a jawbone which includes a tubular x-ray generating unit placed in an oral cavity, and a movable x-ray detection unit placed in the oral cavity outer side region corresponding to the x-ray generating unit and corresponding to a face or an x-ray detection unit having a curved surface shape similar to the face. The x-ray imaging system having the configuration may obtain a teeth x-ray image with a large surface area while minimizing the dose of x-ray exposed to a patient by placing an x-ray generating device in the oral cavity and disposing a movable sensor or a sensor with a large surface area on the outer side of the oral cavity to obtain an x-ray image, reduce excessive x-ray exposure and foreign body sensation in the oral cavity, which a panoramic mode x-ray generating device or an oral sensor-type x-ray generating device in the related art has, to enhance the safety and convenience of the patient, and obtain a clear image with a large surface area, which helps a medical staff in making an exact judgment, with the least number of shootings. An intraoral radiation type x-ray imaging system for capturing an x-ray image of teeth or a jawbone includes an x-ray generating unit to be placed in an oral cavity and an x-ray detection unit corresponding to the x-ray generating unit and to be placed on the outer side of the oral cavity. The x-ray generating unit includes a tubular electron emitting source and x-ray emitting source, the electron emitting source supplying electrons to the x-ray emitting source, and a collimator. The x-ray emitting source emits x-rays at 360.degree. and the collimator regulates the radiation direction of the emitted x-rays to an angle less than 360.degree. The collimator regulates the radiation dose of the x-ray generated.

U.S. Pat. No. 8,119,990 teaches a system for automatic detection of x-rays at an x-ray sensor. A source emits x-ray radiation towards an x-ray sensor, and the x-ray sensor automatically detects the x-ray radiation. The x-ray sensor automatically detects x-ray radiation by evaluating a time series and determining that a voltage threshold is crossed a certain amount of time earlier than the average time it takes the voltage threshold to be crossed from dark current and other noise.

U.S. Pat. No. 6,697,948 teaches a system which enables owners and vendors of software products to protect the property rights of their software, and which utilizes a unique vendor tag system for each instant of a specific software product. This system interacts with a monitoring program, which is running on the user’s device to ensure that no unauthorized use takes place.

Referring to FIG. 5 a media acquisition engine, which U.S. Pat. No. 9,462,082 teaches, includes an interface engine that receives a selection from a plug-in coupled to a media client engine where a client associated with the media client engine identified as subscribing to a cloud application imaging service. The media acquisition engine also includes a media control engine that directs, in accordance with the selection, a physical device to image a physical object and produce a media item based on the image of the physical object, the physical device being coupled to a cloud client. The media acquisition engine further includes a media reception engine that receives the media item from the physical device, and a translation engine that encodes the media item into a data structure compatible with the cloud application imaging service. The interface engine is configured to transfer the media item to the plug-in. Digital imaging has notable advantages over traditional imaging, which processes an image of a physical object onto a physical medium. Digital imaging helps users such as health professionals avoid the costs of expensive processing equipment, physical paper, physical radiographs, and physical film. Techniques such as digital radiography expose patients to lower doses of radiation than traditional radiography and are often safer than their traditional counterparts are. Digital images are easy to store on storage such as a computer’s hard drive or a flash memory card, are easy transferable, and are more portable than traditional physical images. Many digital imaging devices use sophisticated image manipulation techniques and filters that accurately image physical objects. A health professional’s information infrastructures and business processes can potentially benefit from digital imaging techniques. Though digital imaging has many advantages over physical imaging, digital imaging technologies are far from ubiquitous in health offices as existing digital imaging technologies present their own costs. To use existing digital imaging technologies, a user such as a health professional must purchase separate computer terminals and software licenses for each treatment room. As existing technologies install a full digital imaging package on each computer terminal, these technologies are often expensive and present users with more options than they are willing to pay for. Existing digital imaging technologies require users to purchase a complete network infrastructure to support separate medical imaging terminals. Users often face the prospects of ensuring software installed at separate terminals maintains patient confidentiality, accurately stores, and backs up data, accurately upgrades, and correctly performs maintenance tasks. Existing digital imaging technologies are not readily compatible with the objectives of end-users, such as health professionals. Still a networking system 100 for providing one or more application imaging services includes a desktop computer 102, a laptop computer 104, a server 106, a network 108, a server 110, a server 112, a tablet device 114, and a private network group 120. The private network group 120 includes a laptop computer 122, a desktop computer 124, a scanner 126, a tablet device 128, an access gateway 132, a first physical device 134, a second physical device 136, and a third physical device 138. The desktop computer 102, the laptop computer 104, the server 106, the server 110, the server 112, and the tablet device 114 are shown directly connected to the network 108, but can be grouped in a manner similar to the private network group 120 without departing from the scope and substance of the inventive concepts disclosed herein. The desktop computer 102 can include a computer having a separate keyboard, monitor, and processing unit. The desktop computer 102 can integrate one, or more, of the keyboard, the monitor, and the processing unit into a common physical module. The laptop computer 104 can include a portable computer. The laptop 104 can integrate the keyboard, monitor, and processing unit into one physical module. The laptop 104 can also have a battery so that the laptop 104 allows portable data processing and portable access to the network 108. The tablet 114 can include a portable device with a touch screen, a monitor, and a processing unit all integrated into one physical module. Any or all of the computer 102, the laptop 104, and the tablet device 118 can include a computer system. A computer system will usually include a processor, memory, non-volatile storage, and an interface. Peripheral devices can also form a part of the computer system. A typical computer system will include at least a processor, memory, and a device (e.g., a bus) coupling the memory to the processor. The processor can include a general-purpose central processing unit (CPU), such as a microprocessor, or a special-purpose processor, such as a microcontroller. The memory can include a random-access memory (RAM), such as dynamic RAM (DRAM) and static RAM (SRAM). The memory can be local, remote, or distributed. The term “computer-readable storage medium” includes physical media, such as memory. The bus of the computer system can couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. A direct memory access process often writes some of this data into memory during execution of software on the computer system. The non-volatile storage can be local, remote, or distributed. The non-volatile storage is optional because systems need only have all applicable data available in memory. Software is typically stored in the non-volatile storage. Indeed, for large programs, it may not even be possible to store the entire program in memory. Nevertheless, for software to run, if necessary, it is moved to a computer-readable location appropriate for processing, and for illustrative purposes, this paper refers to that location as the memory. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable storage medium.” A processor is “configured to execute a program” when at least one value associated with the program is stored in a register readable by the processor. The bus can also couple the processor to one, or more, interface. The interface can include either a modem or a network interface. Either the modem or the network interface can be part of the computer system. The interface can include an analog modem, ISDN modem, cable modem, token ring interface, satellite transmission interface (e.g. “direct PC”), or other interfaces for coupling a computer system to other computer systems. The interface can include one, or more, input and/or output (I/O) devices. The I/O devices can include a keyboard, a mouse or other pointing device, disk drives, printers, a scanner, and other I/O devices, including a display device. The display device can include a cathode ray tube (CRT), liquid crystal display (LCD), or some other applicable known or convenient display device. An operating system software that includes a file management system, such as a disk operating system, can control the computer system. The operating system software with associated file management system software is the family of operating systems known as Windows® from Microsoft Corporation of Redmond, Wash., and their associated file management systems. Operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage.

An algorithm is here, and, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. The algorithms and displays presented herein do not inherently relate to any computer, or other apparatus. Various general-purpose systems can be used with programs to configure the general-purpose systems in a specific manner. Any, or all, of the computer 102, the laptop 104, and the tablet device 118 can include engines. As used in this paper, an engine includes a dedicated or shared processor and, typically, firmware or software modules that the processor executes. Depending upon implementation-specific or other considerations, an engine can have a centralized distributed location and/or functionality. An engine can include special purpose hardware, firmware, or software embodied in a computer-readable medium for execution by the processor. A computer-readable medium is intended to include all mediums that are statutory (e.g., in the U.S., under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware. Any, or all, of the computer 102, the laptop 104, and the tablet device 118 can include one or more datastores. A datastore can be implemented as software embodied in a physical computer-readable medium on a general- or specific-purpose machine, in firmware, in hardware, in a combination thereof, or in an applicable known or convenient device or system. Datastores in this paper are intended to include any organization of data, including tables, comma-separated values (CSV) files, traditional databases (e.g., SQL), or other applicable known or convenient organizational formats. Datastore-associated components, such as database interfaces, can be considered “part of” a datastore, part of some other system component, or a combination thereof, though the physical location and other characteristics of datastore-associated components is not critical for an understanding of the techniques described in this paper.

Data stores can include data structures. A data structure is associated with a particular way of storing and organizing data in a computer so for efficient use within a given context. Data structures are generally based on the ability of a computer to fetch and store data at any place in its memory, specified by an address, a bit string that can be itself stored in memory and manipulated by the program. Some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure.

The desktop computer 102, the laptop 104, or the tablet device 114 can function as network clients. Any, or all, of the desktop computer 102, the laptop 104, and the tablet device 114 can include one or more operating system software as well as application system software. For instance, the desktop computer 102, the laptop 104, or the tablet device 114 can run a version of a Windows® operating system from Microsoft Corporation, a version of a Mac operating system from Apple Corporation, a Linux based operating system such as an Android operating system, a Symbian operating system, a Blackberry operating system, or other operating system. The desktop computer 102, the laptop 104, and the tablet device 114 can also run one or more applications with which end-users can interact. For instance, the desktop computer 102, the laptop 104, and the tablet device 114 can run word processing applications, spreadsheet applications, imaging applications, and other applications. Any, or all, of the desktop computer 102, the laptop 104, and the tablet device 114 can also run one or more programs that allow a user to access content over the network 108. Any, or all, of the desktop computer 102, the laptop 104, and the tablet device 114 can include one or more web browsers that access information over the network 108 by Hypertext Transfer Protocol (HTTP). The desktop computer 102, the laptop 104, and the tablet device 114 can also include applications that access content via File Transfer Protocols (FTP) or other standards. The desktop computer 102, the laptop 104, or the tablet device 114 can also function as servers. A server is an electronic device that includes one or more engines dedicated in whole or in part to serving the needs or requests of other programs and/or devices. The desktop computer 102, the laptop 104, or the tablet device 114 can distribute data and/or processing functionality across the network 108 to facilitate providing cloud application imaging services. Any of the server 106, the server 110, and the server 112 can include computer systems. Any of the server 106, the server 110, and the server 112 can include one or more engines. Any of the server 106, the server 110, and the server 112 can incorporate one or more datastores. The engines in any of the server 106, the server 110, and the server 112 can be are dedicated in whole or in part to serving the needs or requests of other programs and/or devices. Any of the server 106, the server 110, and the server 112 can handle relatively high processing and/or memory volumes and relatively fast network connections and/or throughput. The server 106, the server 110, and the server 112 may or may not have device interfaces and/or graphical user interfaces (GUIs). Any of the server 106, the server 110, and the server 112 can meet or exceed high availability standards. The server 106, the server 110, and the server 112 can incorporate robust hardware, hardware redundancy, network clustering technology, or load balancing technologies to ensure availability. The server 106, the server 110, and the server 112 can incorporate administration engines that from electronic devices such as the desktop computer 102, the laptop computer 104, or the tablet device 114, or other devices access remotely through the network 108. Any of the server 106, the server 110, and the server 112 can include an operating system that is configured for server functionality, i.e., to provide services relating to the needs or requests of other programs and/or devices. The operating system in the server 106, the server 110, or the server 112 can include advanced or distributed backup capabilities, advanced or distributed automation modules and/or engines, disaster recovery modules, transparent transfer of information and/or data between various internal storage devices as well as across the network, and advanced system security with the ability to encrypt and protect information regarding data, items stored in memory, and resources. The server 106, the server 110, and the server 112 can incorporate a version of a Windows® server operating system from Microsoft Corporation, a version of a Mac server operating system from Apple Corporation, a Linux based server operating system, a UNIX based server operating system, a Symbian server operating system, a Blackberry server operating system, or other operating system. The server 106, the server 110, and the server 112 can distribute functionality and/or data storage. For instance, the server 106, the server 110, and the server 112 can distribute the functionality of an application server and can run different portions of one or more applications concurrently. In such a case, each of the server 106, the server 110, and the server 112 stores and/or executes distributed portions of application services, communication services, database services, web and/or network services, storage services, and/or other services. The server 106, the server 110, and the server 112 can distribute storage of different engines or portions of engines. The networking system 100 can include the network 108. The network 108 can include a networked system that includes several computer systems coupled, such as a local area network (LAN), the Internet, or some other networked system. The term “Internet” as used in this paper refers to a network of networks that uses certain protocols, such as the TCP/IP protocol, and possibly other protocols such as the HTTP for hypertext markup language (HTML) documents that make up the World Wide Web (the web). Content servers, which are “on” the Internet, often provide the content. A web server, which is one type of content server, is typically at least one computer system, which operates as a server computer system, operates with the protocols of the World Wide Web, and has a connection to the Internet. Applicable known or convenient physical connections of the Internet and the protocols and communication procedures of the Internet and the web are and/or can be used. The network 108 can broadly include, as understood from relevant context, anything from a minimalist coupling of the components to every component of the Internet and networks coupled to the Internet. Components that are outside of the control of the networking system 100 are sources of data received in an applicable known or convenient manner. The network 108 can use wired or wireless technologies, alone or in combination, to connect the devices inside the networking system 100. Wired technologies connect devices using a physical cable such as an Ethernet cable, digital signal link lines (T1-T3 lines), or other network cable. The private network group 120 can include a wired local area network wired personal area network (PAN), a wired LAN, a wired metropolitan area network, or a wired wide area network. Some, or all, of the network 108 can include cables that facilitate transmission of electrical, optical, or other wired signals. Some, or all, of the network 108 can also employ wireless network technologies that use electromagnetic waves at frequencies such as radio frequencies (RF) or microwave frequencies. The network 108 can include transmitters, receivers, base stations, and other equipment that facilitates communication via electromagnetic waves. Some, or all, of the network 108 can include a wireless personal area network (WPAN) technology, a wireless local area network (WLAN) technology, a wireless metropolitan area network technology, or a wireless wide area network technology. The network 108 can use Global System for Mobile Communications (GSM) technologies, personal communications service (PCS) technologies, third generation (3G) wireless network technologies, or fourth generation (4G) network technologies. The network 108 can also include all, or portions, of a Wireless Fidelity (Wi-Fi) network, a Worldwide Interoperability for Microwave Access (WiMAX) network, or other wireless networks. The networking system 100 can include the private network group 120. The private network group 120 is a group of computers that form a subset of the larger network 108. The private network group 120 can include the laptop computer 122, the desktop computer 124, the scanner 126, the tablet device 128, the access gateway 132, the first physical device 134, the second physical device 136, and the third physical device 138. The laptop computer 122 can be similar to the laptop computer 104, the desktop computer 124 can be similar to the desktop computer 102, and the tablet device 128 can be similar to the tablet device 114. Any of the laptop computer 122, the desktop computer 124, the scanner 126, the tablet device 128, the access gateway 132, the first physical device 134, the second physical device 136, and the third physical device 138 can include computer systems, engines, datastores. Any of the laptop computer 122, the desktop computer 124, the scanner 126, the tablet device 128, the access gateway 132, the first physical device 134, the second physical device 136, and the third physical device 138 can incorporate components similar to the components in a networking system. The private network group 120 can include a private network. A private network provides a set of private internet protocol (IP) addresses to each of its members while maintaining a connection to a larger network, here the network 108. To this end, the members of the private network group 120 (i.e., the laptop computer 122, the desktop computer 124, the scanner 126, the tablet device 128, the first physical device 134, the second physical device 136, and the third physical device 138) can each be assigned a private IP address irrespective of the public IP address of the router 132. Though the term “private” appears in conjunction with the name of the private network group 120, the private network group 120 can actually include a public network that forms a subset of the network 108. In such a case, each of the laptop computer 122, the desktop computer 124, the scanner n 126, the tablet device 128, the first physical device 134, the second physical device 136, and the third physical device 138 can have a public IP address and can maintain a connection to the network 120. The connection of some or all of the laptop computer 122, the desktop computer 124, the scanner 126, the tablet device 128, the first physical device 134, the second physical device 136, and the third physical device 138 can be a wired or a wireless connection. The private network group 120 can include the access gateway 132. The access gateway 132 assigns private IP addresses to each of the devices 122, 124, 126, 128, 134, 136, and 138. The access gateway 132 can establish user accounts for each of the devices 122, 124, 126, 128, 134, 136, and 138 and can restrict access to the network 108 based on parameters of those user accounts. The access gateway 132 can also function as an intermediary to provide content from the network 108 to the devices 122, 124, 126, 128, 134, 136, and 138. The access gateway 132 can format and appropriately forward data packets traveling over the network 108 to and from the devices 122, 124, 126, 128, 134, 136, and 138. The access gateway 132 can be a router, a bridge, or other access device. The access gateway 132 can maintain a firewall to control communications coming into the private network group 120 through the network 108. The access gateway 132 can also control public IP addresses associated with each of the laptop computer 122, the desktop computer 124, the scanner 126, the tablet device 128, the first physical device 134, the second physical device 136, and the third physical device 138. The access gateway 132 is absent and each of the devices inside the private network group 120 can maintain its own connection to the network 108. The desktop computer 124 is shown connected to the access gateway 132 as such a configuration is a common implementation. However, the functions described in relation to the desktop computer 124 can be implemented on the laptop computer 122, the tablet device 128, or any applicable computing device. The private network group 120 can be located inside a common geographical area or region. The private network group 120 may be located at a school, a residence, a business, a campus, or other location. The private network group 120 is located inside a health office, such as the office of a dentist, a doctor, a chiropractor, a psychologist, a veterinarian, a dietician, a wellness specialist, or other health professional. The physical devices 134, 136, and 138 can image a physical object. The physical devices 134, 136, and 138 can connect to the desktop computer 124 via a network connection or an output port of the desktop computer 124. Similarly, the physical devices 134, 136, and 138 can connect to the laptop computer 122, the tablet device 128, or a mobile phone. The physical devices 134, 136, and 138 are directly connected to the access gateway 132. The physical devices 134, 136, and 138 can also internally incorporate network adapters that allow a direct connection to the network 108. The first physical device 134 can be a sensor-based imaging technology. A sensor is a device with electronic, mechanical, or other components that measures a quantity from the physical world and translates the quantity into a data structure or signal that a computer, machine, or other instrument can read. The first physical device 134 can use a sensor to sense an attribute of a physical object. The physical object can include, for instance, portions of a person’s mouth, head, neck, limb, or other body part. The physical object can be an animate or inanimate item. The sensor can include x-ray sensors to determine the boundaries of uniformly or non-uniformly composed material such as part of the human body. The sensor can be part of a Flat Panel Detector (FPD). Such an FPD can be an indirect FPD including amorphous silicon or other similar material used along with a scintillator. The indirect FPD can allow the conversion of x-ray energy to light, which is eventually translated into a digital signal. Thin Film Transistors (TFTs) or Charge Coupled Devices (CCDs) can subsequently allow imaging of the converted signal. Such an FPD can also be a direct FPD that uses Amorphous Selenium or other similar material. The direct FPD can allow for the direct conversion of X-ray photons to charge patterns that, in turn, are converted to images by an array such as a TFT array, an Active Matrix Array, or by Electrometer Probes and/or Microplasma Line Addressing. The sensor can also include a High-Density Line Scan Solid State detector. The sensor of the first physical device 134 can include an oral sensor. An oral sensor is a sensor that a user such as a health practitioner can insert into a patient’s mouth. The first physical device 134 can reside in a dentist’s office that operates the private network group 120. The sensor of the first physical device 134 can also include a sensor that is inserted into a person’s ear, nose, throat, or other part of a person’s body. The second physical device 136 can include a digital radiography device. Radiography uses X-rays to view the boundaries of uniformly or non-uniformly composed material such as part of the human body. Digital radiography is the performance of radiography without the requirements of chemical processing or physical media. Digital radiography allows for the easy conversion of an image to a digital format. The digital radiography device can be located in the office of a health professional. The third physical device 138 can include a thermal-based imaging technology. Thermal imaging technology is technology that detects the presence of radiation the infrared ranges of the electromagnetic spectrum. Thermal imaging technology allows the imaging of the amount of thermal radiation emitted by an object. The third physical device 138 can include an oral sensor, or a sensor that is inserted into a person’s ear, nose, throat, or other part of a person’s body. The third physical device 138 resides in the office of a health professional, such as the office of a dentist, a doctor, a chiropractor, a psychologist, a veterinarian, a dietician, a wellness specialist, or other health professional. An office can employ one or more of the first physical device 134, the second physical device 136, and the third physical device 138 alone or in combination. Each of the first physical device 134, the second physical device 136, and the third physical device 138 can reside in a general-purpose computer, such as the desktop computer 124, the tablet device 128, the laptop computer 122, and/or a mobile phone. The networking system 100 can facilitate delivery of n a cloud application imaging service. A cloud application imaging service is a service that allows an entity associated with a physical device (such as one of the physical devices 134, 136, and 138) to use a cloud-computing application that is executed on a client computer (such as the desktop computer 124) to direct the physical device to image a physical object. Cloud-based computing, or cloud computing, is a computing architecture in which a client can execute the full capabilities of an application in a container, such as a web browser. Though the application executes on the client, portions of the application can be distributed at various locations across the network. Portions of the cloud application imaging service that are facilitated by the networking system 100 can reside on one or more of the desktop computer 102, the laptop computer 104, the server 106, the server 110, the server 112, the tablet device 114, and/or other locations “in the cloud” of the networking system 100. The application can appear as a single point of access for an end-user using a client device such as the desktop computer 124. The cloud application imaging service can implement cloud client functionalities onto the desktop computer 124. A cloud client incorporates hardware and/or software that allows a cloud application to run in a container such as a web browser. Allowing the desktop computer 124 to function as a cloud client requires the presence of a container in which the cloud application imaging service can execute on the desktop computer 124. The cloud application imaging service can facilitate communication over a cloud application layer between the client engines on the desktop computer 124 and the one or more server engines on the desktop computer 102, the laptop computer 104, the server 106, the server 110, the server 112, the tablet device 114, and/or other locations “in the cloud” of the networking system 100. The cloud application layer or “Software as a Service” (SaaS) facilitates the transfer over the Internet of software as a service that a container, such as a web browser, can access. The desktop computer 124 need not install the cloud application imaging service even though the cloud application imaging service executes on the desktop computer 124. The cloud application imaging service can also deliver to the desktop computer 124 one or more Cloud Platform as a Service (PaaS) platforms that provide computing platforms, solution stacks, and other similar hardware and software platforms. The cloud application imaging service can deliver cloud infrastructure services, such as Infrastructure as a Service (laaS) that can virtualize and/or emulate various platforms, provide storage, and provide networking capabilities. The cloud application imaging service, consistent with cloud-computing services in general, allows users of the desktop computer 124 to subscribe to specific resources that are desirable for imaging and other tasks related to the physical devices 134, 136, and 138. Providers of the cloud application imaging service can bill end-users on a utility computing basis and can bill for use of resources. In the health context, providers of the cloud application imaging service can bill for items such as the number of images an office wishes to process, specific image filters that an office wishes to use, and other use-related factors.

Referring to FIG. 6 a universal image capture manager (UICM), which U.S. Pat. Application Publication No. 2011/0304740 teaches, facilitates the acquisition of image data from a plurality of image source devices (ISDs) to an image utilizing software application (IUSA). The UICM is implemented on a computer processing device and includes a first software communication interface configured to facilitate data communication between the UICM and an IUSA. The UICM also includes a translator/mapper (T/M) software component being in operative communication with a first software communication interface and configured to translate and map an image request from an IUSA to at least one device driver (DD) software component of a plurality of DD software components. The UICM further includes a plurality of DD software components being in operative communication with the T/M software component. Each of the DD software components is configured to facilitate data communication with at least one ISD. Many times, it is desirable to bring images into a user software application. This is often done in the context of a medical office environment or a hospital environment. Images may be captured by image source devices such as a digital camera device or an X-ray imaging device and are brought into a user software application such as an imaging software application or a practice management software application running on a personal computer or a workstation.

Each image source device may require a different interface and image data format for acquiring image data from that image source device. The various interfaces may be TWAIN-compatible or not, maybe in the form of an application program interface (API), a data link library (DLL), or some other type of interface. Also, the various image data may be raw image data, DICOM image data, 16-bit or 32-bit or 64-bit image data, or some other type of image data. The process of acquiring an image into a user software application can be difficult and cumbersome. To acquire and place an image in a user software application, a user may have to first leave the software application, open a hardware driver, set the device options, acquire the image, save the image to a local storage area, close the hardware driver, return to the software application, locate the saved image, and read the image file from the local storage area. Hardware and software developers have developed proprietary interfaces to help solve this problem. Having many proprietary interfaces has resulted in software developers having to write a driver for each different device to be supported. This has also resulted in hardware device manufacturers having to write a different driver for each software application. General interoperability between user software applications and image source devices has been almost non-existent.

Still referring to FIG. 6 a system 100 for acquiring image data from multiple sources includes an image utilizing software application (IUSA) 110 implemented on a first computer processing device 111, a universal image capture manager (UICM) 120 implemented on a second computer processing device 121, and a plurality of image source devices (ISDs) 130 (e.g., ISD #1 to ISD #N, where N represents a positive integer). The IUSA 110 may be a client software application such as an imaging software application or a practice management application as may be used in a physician’s office, a dentist’s office, or a hospital environment. The IUSA 110 is implemented on the first computer processing device 111, such as a personal computer (PC) or a workstation computer. The plurality of ISDs 130 are hardware-based devices that are capable of capturing images in the form of image data (e.g., digital image data). ISDs may include a visible light intra-oral camera, an intra-oral x-ray sensor, a panoramic (pan) x-ray machine, a cephalometric (ceph) x-ray machine, a scanner for scanning photosensitive imaging plates, and a digital endoscope. There exist many types of ISDs using many diverse types of interfaces and protocols to export the image data from the ISDs. The universal image capture manager (UICM) 120 is a software application or a software module. The second computer processing device 121, having the UICM 120, operatively interfaces between the first computer processing device 111, having the IUSA 110, and the plurality of ISDs 130, and acts as an intermediary between the IUSA 110 and the plurality of ISDs 130. The UICM 120 is a software module implemented on the second computer processing device 121 such as a personal computer (PC), a workstation computer, a server computer, or a dedicated processing device designed specifically for UICM operation.

The UICM 120 is configured to communicate in a single predefined manner with the IUSA 110 to receive image request messages from the IUSA 110 and to return image data to the IUSA 110. The UICM 120 is configured to acquire image data from the multiple image source devices (ISDs) 130. As a result, the IUSA 110 does not have to be concerned with being able to directly acquire image data from multiple different image data sources itself. Instead, the UICM 120 takes on the burden of communicating with the various ISDs 130 with their various communication interfaces and protocols.

U.S. Pat. No. 10,610,189 teaches an x-ray irradiating device having a function of selecting an automatic radiographic mode which includes an x-ray source, a motion sensor configured to detect an x-ray source pointing direction, and a controller configured to select any one of a plurality of radiographic modes according to a pointing angle signal received from the motion sensor. An x-ray imaging method includes the steps of setting a reference plane as a reference value for a pointing angle in which an x-ray source is directed, detecting the pointing angle relative to the reference plane by using a motion sensor and selecting a radiographic mode corresponding to the detected pointing angle, and performing a radiography by controlling the x-ray source with a set value corresponding to the selected radiographic mode.

X-ray imaging is a radiography method using permeability of x-rays and acquires an x-ray image of an internal structure of a subject to be imaged based on the attenuation that is accumulated in the process of the x-rays passing through the subject. To achieve this, an x-ray imaging apparatus includes an x-ray irradiating device emitting x-rays, an x-ray sensor disposed to face the x-ray irradiating device with a subject therebetween and configured to receive the x-rays having passed through the subject; and an image processor configured to produce an x-ray image by using a detection result of the x-ray sensor. The x-ray irradiating device has been continuously reduced in size and improved in convenience. Recently, compact x-ray irradiating devices, which can be easily used by radiographers, have been frequently used for diagnosis in a hospital or nondestructive examination in an industrial field. This compact x-ray irradiating device is also used for intraoral radiography in the dental field. When it is required to perform quick radiography for an examinee having difficulty in moving during an implant procedure or the like, a hand-held type x-ray irradiating device or a compact x-ray irradiating device connected to an instrument called as a standard arm is used. During intraoral radiography, a compact x-ray sensor or a film is inserted into an examinee’s mouth, and a radiographer performs radiography by irradiating x-rays toward the inserted x-ray sensor while holding the x-ray irradiating device by hand. In performing intraoral radiography, it is necessary to adjust the dose of x-ray radiation according to the position and size of teeth, etc. of a subject, the size of an examinee’s body, and the like. To achieve this, the radiographer performs a process of changing the setting of the x-ray irradiating device through an input means including a button or a touch interface before radiography. In this case, the radiographer sets the radiographic mode with one hand while holding the x-ray irradiating device with the other hand in the state where the intraoral x-ray sensor is inserted into the examinee’s mouth. This process causes inconvenience in both the examinee with the x-ray sensor inserted into the mouth while waiting and the radiographer manipulating the input means while holding the heavy x-ray irradiating device with one hand.

The applicants hereby incorporate the above referenced patent and patent application publications into their specification.

### SUMMARY OF THE INVENTION

The present invention is a sensor licensing system for an intraoral sensor which includes an on/off mechanism, a driver, a sensor and a sensor memory. The on/off mechanism is coupled to the intraoral sensor and has an encrypted licensing code so that the on/off mechanism turns on and off the intraoral sensor.

In the first aspect of the present invention the driver processes the encrypted license code stored in the sensor memory and determines if license code is expired based on the computer clock, exposure counter or other desired limits.

In the second aspect of the present invention the sensor periodically checks an online server for an updated encrypted license code and stores new license code in the sensor memory.

In the third aspect of the present invention the updated license code is created and stored on the server when a new periodic subscription payment is made.

In the fourth aspect of the present invention the control center periodically checks and downloads any updated license code.

In the fifth aspect of the present invention a failure to make a payment will result in no encrypted license code being created and eventual expiration of the old, encrypted license code.

In the sixth aspect of the present invention the encrypted licensing code incorporates date and image counter checking to prevent tampering.

In the seventh aspect of the present invention the sensor licensing system includes a mechanism of detecting potential expiration of sensor license and notifying user of need to either update license or connect sensor to a server to check/update licensing status.

In the eighth aspect of the present invention the driver controls operation of the intraoral sensor on a computer and the software operates on the computer not only in real time, but also in the background so that the control center controls image acquisition and settings for the intraoral sensor so that it can be used with almost any dental imaging software on the market.

In the ninth aspect of the present invention the control center also monitors performance of the intraoral sensor during use and notifies the user of problems.

In the tenth aspect of the present invention the control center acts as the primary driver for the intraoral sensor and interfaces with other imaging applications to acquire and serve up images for processing and display wherein the dental image acquisition software interfaces with other applications via either an SDK driver or a TWAIN driver whereby the control center can perform preprocessing on the image before transferring to an imaging application, including but not limited noise filtering, sharpening, contrast enhancement or histogram equalization.

In the eleventh aspect of the present invention a dose optimization tool for an intraoral sensor guides user to perform initial exposure measurements of an x-ray head for optimal signal level to minimize patient exposure to ionized radiation to insure optimal image quality while insuring that the lowest possible x-ray generator settings to achieve diagnosable x-ray images.

In the twelfth aspect of the present invention the dose optimization tool recommends ideal dose/time settings for use based on adult/child and different x-ray energies.

In the thirteenth aspect of the present invention the dose optimization tool includes a real-time signal monitor which monitors image level and will continue to monitor exposure of every image and notify the operator if signal is too high or low immediately after images acquisition during clinical use.

In the fourteenth aspect of the present invention the dose monitor of the dose optimization tool operates in the background thereby allowing it to be used independent of the imaging application software being used.

In the fifteenth aspect of the present invention the exposure monitor counts sensor usage and records data such as total exposures.

In the sixteenth aspect of the present invention the control center saves and stores recent images to allow for remote review and diagnosis of sensor issues during debugging.

In the seventeenth aspect of the present invention the control center provides integrated support which is live with built-in support tools operating in the background, independent of the imaging application.

In the eighteenth aspect of the present invention the control center monitors real time for software or sensor firmware updates.

In the nineteenth aspect of the present invention the control center is a system and method for licensing of software applications over a network.

Other aspects and many of the attendant advantages will be more readily appreciated as the same becomes better understood by reference to the following detailed description and considered in connection with the accompanying drawing in which like reference symbols designate like parts throughout the figures.

The features of the invention which are believed to be novel are set forth with particularity in the appended claims.

### DESCRIPTION OF THE DRAWINGS

FIG. 1 is a schematic diagram of a dental x-ray system including an x-ray source, an intraoral sensor located in a patient’s mouth and a computer connected to the intraoral sensor according to U.S. Pat. No. 9,259,197.

FIG. 2 is an exploded perspective drawing of an intraoral sensor which has electronic components, both in its head side and its tail side, for operating a control center according to the present invention.

FIG. 3 is a schematic diagram of electronic components both at the intraoral sensor’s head side consisting of an Analog Front End (AFE) IC, a FGPA, clock drivers, a Flash memory, and a power regulation circuit.

FIG. 4 is a schematic diagram of electronic components both at the intraoral sensor’s head side and at the USB’s tail side consisting of a USB controller IC, a FPGA, Flash memory, SDRAM, and power regulation circuits.

FIG. 5 is a conceptual schematic diagram of a networking system according to U.S. Pat. No. 9,462,082.

FIG. 6 is a schematic block diagram of a system for acquiring image data from multiple sources according to U.S. Pat. Application Publication No. 2011/0304740.

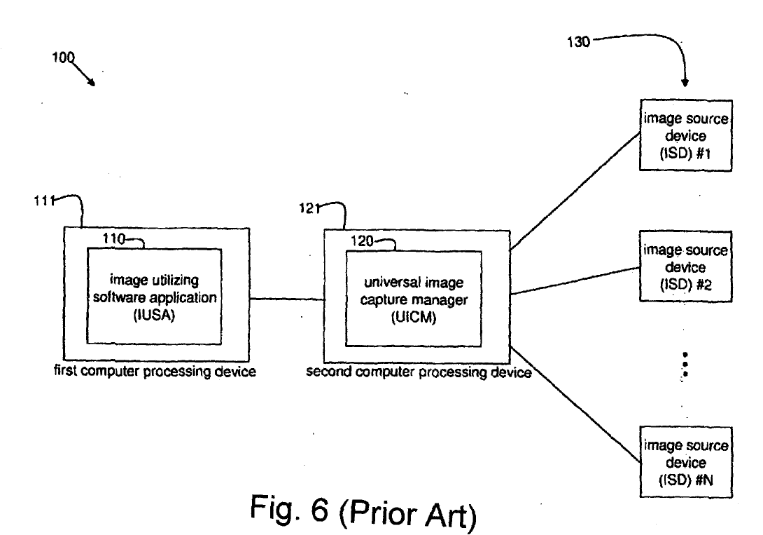

FIG. 7 is a schematic diagram of a control center which manages licenses, licensing data, tamper protection, x-ray dose optimization, exposure monitoring and other information for use with the intraoral sensor of FIG. 2 according to the present invention.

FIG. 8 is an overview of the involved parties required to handle the x-ray sensor license distribution and enforcement in the form of a schematic drawing of a licensing management system of the control center of FIG. 7 which includes a plurality of control centers, a plurality of intraoral sensors, an FTP license server, a license database server with a memory containing licenses, a licensing client and a payment system according to the present invention.

FIG. 9 is a diagram of the interaction of at least one of the control centers, at least one of the intraoral sensors, the FTP license server, a license database server with a memory containing licenses, the licensing client and the payment system of the licensing management system of FIG. 8.

### DESCRIPTION OF THE PREFERRED EMBODIMENT

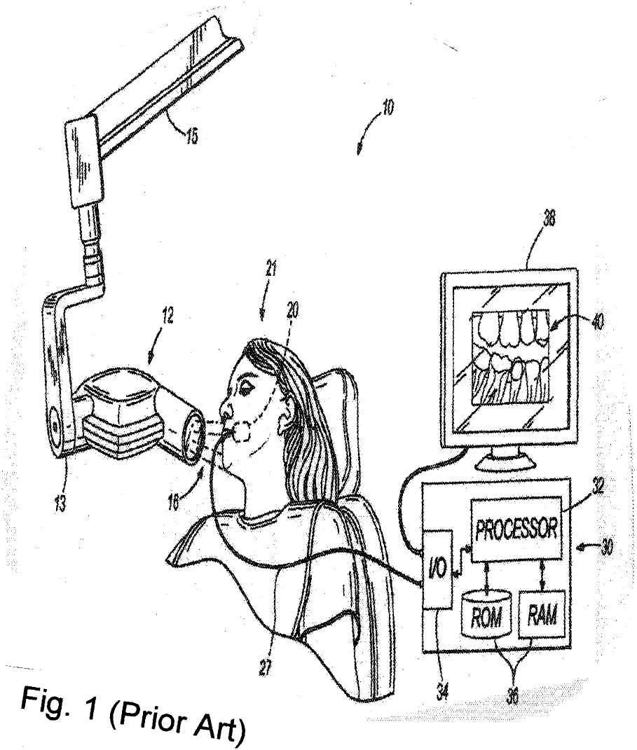

Referring to FIG. 1 a dental x-ray system 10, which U.S. Pat. No. 9,259,197 teaches, includes an x-ray source 12. The x-ray source 12 is located on an end 13 of a mechanical arm 15. When activated, the x-ray source 12 generates an x-ray stream 16 that has a circular cross-section. The x-ray source 12 is positioned by an operator so that the x-ray stream 16 is directed to an intraoral sensor 20. Intraoral sensor 20 is placed in the mouth of patient 21. The intraoral sensor 20 may include a scintillator that converts x-ray radiation to visible light. The intraoral sensor 20 is configured to convert x-rays into electric charge, which in turn are converted to digital signals. These digital signals are provided to a processor 32 which is connected to memory 36, ROM and RAM, and an input-output interface 34. Image data captured by the intraoral sensor 20 and processed by the computer 30 is sent to a display 38 and viewed as image 40.

Still referring to FIG. 1 in the dental x-ray system 10 capturing an image depends on at least two factors activation of the x-ray source 12 and “activation” of the intraoral sensor 20. What constitutes “activation” of the intraoral sensor 20 can vary based upon the type of intraoral sensor 20 used, but in most cases “activation” occurs when the intraoral sensor 20 detects the x-ray exposure and then automatically starts image capture which is the integration of the image. It would be advantageous if the automatic “activation” of the intraoral sensor 20 could be prohibited depending on the licensing status.

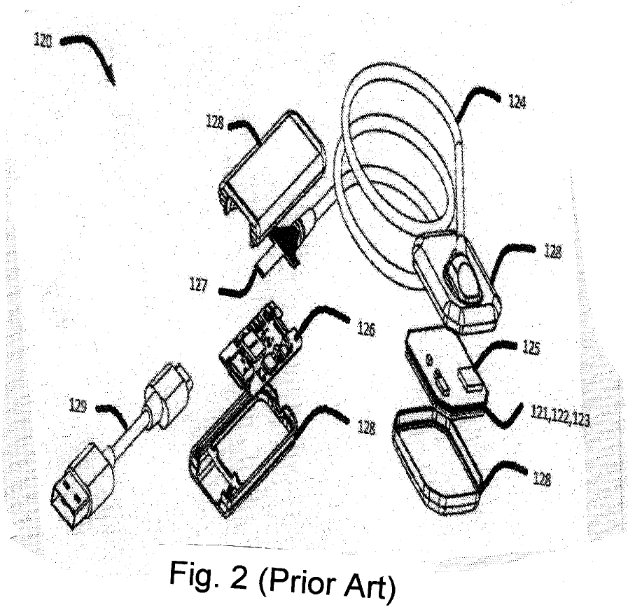

Referring to FIG. 2 critical components for the intraoral sensor and their functions are a CMOS Sensor, a Scintillator, a Fiber Optic, Sensor PCB, a Cable, a Tail PCB. The intraoral sensor is a CMOS imager containing pixels that convert visible photons into electrons. The Scintillator converts x-ray energy into visible photons and is deposited directly onto the fiber optic. The Fiber Optic directs visible photons to the CMOS sensor and blocks most of the direct x-ray energy that passes through the scintillator. The intraoral sensor’s PCB has electronics that generates voltages and timing to detect the onset of x-ray/light, begins CMOS integration and converts charge from the CMOS Sensor during readout into digital signals. The Cable is a multi-conductor cable that transmits digital signals from the intraoral sensor’s PCB to the Tail PCB. The Tail PCB has electronics that receive digital data from the intraoral sensor’s PCB and transfers it to a host computer USB interface. The Connector provides a USB, Type A connection to communicate with the host computer. The Housings is a set of hermetically sealed, plastic parts that protect sensor components from mechanical and environmental conditions. The fiber optic is attached to the CMOS sensor with optical epoxy. The CMOS sensor is glued to the Sensor PCB with epoxy and electrically connected with aluminum wire-bonds. A flex circuit connects the Sensor PCB to the cable. Conductors on the opposite end of the cable are soldered to the Tail PCB. The cable is a custom cable made of 13 wires made up of 2 twisted pairs, 8 single conductors, and 1 ground conductor all within a shielded braid. The cable diameter is 3.0 mm. The cable jacket material is polyurethane.

The intraoral sensor has an x-ray imager (CMOS) that creates a digital image from x-ray doses perceptible by the sensor. The digital image created is immediately visible on the screen of a personal computer connected to the intraoral sensor through the standard USB port. With imaging software, acquired images can be optimized for specific diagnostic tasks, archived as image files, and printed out on a suitable printer.

The intraoral sensor utilizes standard USB 2.0, with 5.0±0.5 V VDC input per the USB specification. The sensor typically draws less than 200 m (about 656.17 ft)A current with a maximum of 350 mA. The intraoral sensor can filter power supply noise of up to 50 mV peak to peak and operate within these levels. The Intraoral sensor can operate continuously.

Software provides utilities for optimizing viewing and printing of images. The intraoral sensor captures x-ray images suitable for recognition of normal anatomical structures, dental pathologies, and abnormal conditions. Inadequate images may result in misdiagnosis, subjecting the patient to incorrect or unnecessary dental procedures that would present an unacceptable risk to the patient.

The intraoral sensor may be aligned with the x-ray source for imaging the desired anatomy. Inadequate alignment may result in repeated x-ray exposures thereby subjecting the patient to additional ionizing radiation that would present an unacceptable risk to the patient. The intraoral sensor can be used either in combination with special positioning devices to facilitate positioning and alignment with the x-ray beam or it may also be positioned by hand with the assistance of the patient. There is no electrical or physical connection between the intraoral sensor and the x-ray generator.

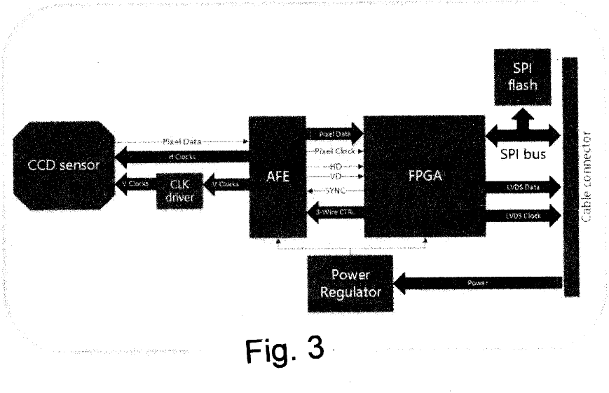

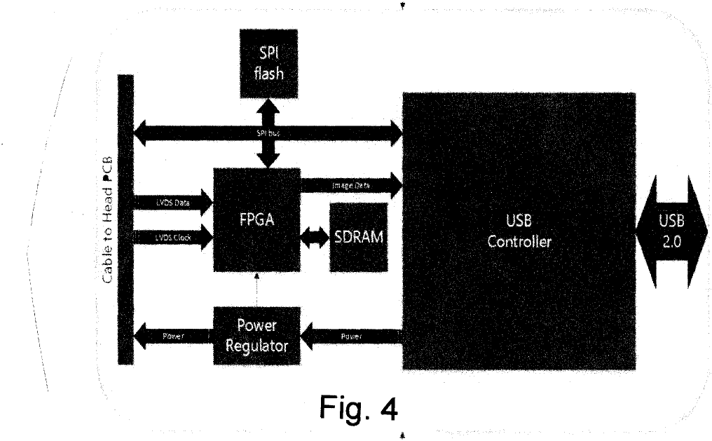

Referring to FIG. 3 in conjunction with FIG. 4, the overall system has electronic components both at the intraoral sensor’s head side and at the USB’s tail side. The major components in the intraoral sensor’s head side consists of an Analog Front End (AFE) IC, a FGPA, clock drivers, Flash memory, and power regulation circuits. The major components in the USB Tail side consists of USB controller IC, a FPGA, Flash memory, SDRAM, and power regulation circuits. The sensor image is readout in an alternating row readout scheme (top row, bottom row, top row, bottom row). After the last row is readout, the sensor goes back to the flush mode waiting for the next detection trigger. In the intraoral sensor’s head electronics, the analog CMOS output feeds into the Analog Front-End (AFE) which controls the CMOS timing and converts the data into 12-bit digital pixel data. From there the data is serialized and is sent to the USB Tail board. The USB tail board receives the serialized image data from the intraoral sensor’s headboard via the LVDS clock and data lines and converts this into image/pixel data. The image data is sent into a dedicated FIFO “channel” in the USB controller and will eventually be read by the PC-side software. The USB Tail board also has additional SDRAM to buffer at least one image if the PC is unable to receive the image data immediately. However, this is only set up as an option and normal operation will have the data directly downloaded to the PC.

The intraoral sensor’s operations with x-ray exposure in the range of 50-70 kV. The steps to achieve an exposure are as follows:

The intraoral sensor is initialized when plugged into a computer. The intraoral sensor remains inactive until armed, at which point the intraoral sensor continuously monitors for x-ray in the form of visible photons. When photons are detected the intraoral sensor integrates for a fixed duration of 750 ms and then begins image readout through the electronics and to the computer. The readout time will depend on USB bandwidth with the host computer but is nominally 2.2 seconds. After readout, the intraoral sensor re-arms itself within 1 ms as it restarts the cycle. The x-ray absorber is Cesium Iodide, deposited directly onto a 1.5 mm thick fiber optic faceplate with packed 6 um fibers and interstitial light absorbing fibers for improved contrast. The scintillator contains a reflector to direct light into the CMOS Sensor. The scintillator and reflector are covered with layers of parylene to protect them from the environment. The energy conversion mechanism is a set of processes that involve energy conversion (x-ray to visible to signal).

In Step 1 an x-ray interacts within scintillator and x-ray energy is generated by the source. After penetrating the patient anatomy, housing and cushion, the x-ray energy hits the scintillator. The x-ray photons interact with the scintillator and some of them are converted into visible photons. The x-ray absorption in the scintillator is a function of x-ray energy and the scintillator thickness.

In Step 2 a Visible light photons existing scintillator. The visible photons generated from the scintillator are collimated by the natural crystal structure of deposited Csl. A light reflector layer on the top of the scintillator reflects light back into the crystal structure and to the CMOS sensor.

In Step 3 the Visible light is focused by the Fiber Optic and enters the top fiber optic surface, collimated by the Csl structure. Light not collimated by Csl as well as light undergoing crosstalk within the fiber optic is absorbed by interstitial absorbers within the fiber optic. This absorption increases overall scintillator/FOP modulation transfer function (MTF).

In Step 4 the CMOS Imager converts light into an electrical signal. The CMOS imager consists of an array of pixels with a fill factor of 1.0, which describes the ratio between the photon sensitive area to the pixel total area. The CMOS imager converts light exiting the fiber optic into electrical charge.

The CMOS Sensor, when active, continuously detects X-ray events by flushing the entire CMOS array. During flushing, the electronics monitors for increases in electrical charge caused by visible photons. The detection method also flushes built up charge the CMOS array that naturally occurs. When detection is made, the sensor resets and begins integrating ongoing visible photons. The time to detect visible photons and begin integration has been measured to be less than 1 ms. After the Integration state, the system automatically transitions into the CMOS Readout state. The full CMOS Pixel Array will be readout by the AFE and transferred to the Tail board. The Tail board will forward the de-serialized Pixel Data onto the USB controller FIFO and the PC application will be able to acquire the image. After the CMOS readout the camera automatically switches the AFE back to Sweep Mode and the camera returns to the Idle state. The data clock is 12 Mhz and a full frame is readout within 2.83 seconds.

Referring to FIG. 2 an intraoral sensor 120 which includes an imaging sensor 121, a fiber optic 122, a scintillator 121, a sensor printed circuit board (Head side PCB) 125, a multi-conductor cable 124, an interface printed circuit board (Tail side PCB) 126, a connector 127 and housings 128. The imaging sensor 121 converts x-ray energy into digital signals. The multi-conductor cable transmits digital signals from the Head side PCB 125 to the Tail side PCB 126. The Tail side PCB 126 includes electronics that receives digital data from the Head side PCB 125 and transfers it to a host computer USB interface. A Type C USB connector 129 communicates with a host computer via replaceable USB-C to USB-A extension cable. 128 are a set of hermetically sealed, plastic parts that protect sensor components from mechanical and environmental conditions.

Still referring to FIG. 2, the major components in the Tail side PCB consists of USB controller IC, a FPGA, a flash memory, and power regulation circuits. In the sensor head electronics, digital imager data is serialized and is sent to the Tail board. Communication and data transfer from the sensor to the host computer is handled by a controller chip located in the tail PCB and handles communication via a USB protocol. Some/all functions located in the Tail Side may alternatively be housed on the Sensor Head.

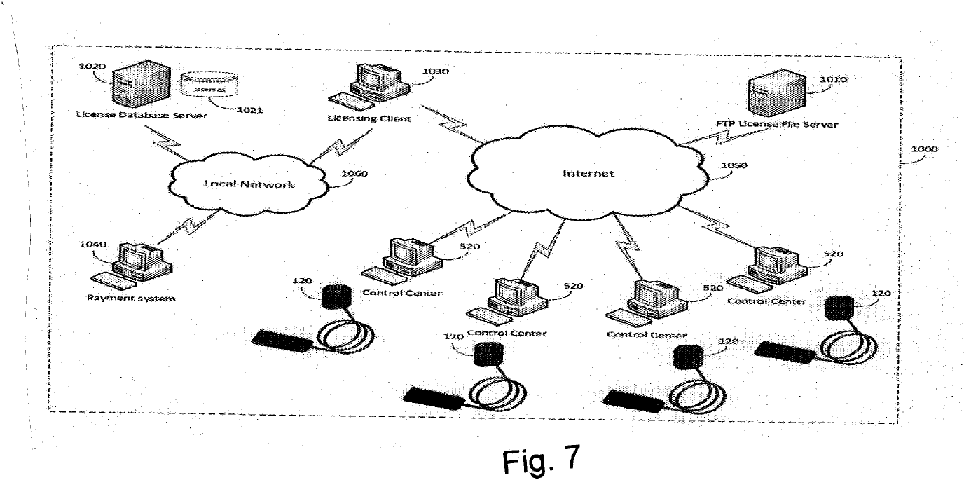

Referring to FIG. 7, a control center and licensing management system for an intraoral sensor includes a sensor licensing system and a dose optimization tool. The sensor licensing system includes an on/off mechanism and a driver. The on/off mechanism is coupled to the intraoral sensor. The driver is coupled to the on/off mechanism. The on/off mechanism has an encrypted licensing code so that the on/off mechanism turns on and off the intraoral sensor.

Still referring to FIG. 7 the driver is dental image acquisition software which acts as the primary driver for the intraoral sensor and interfaces with other imaging applications to acquire and serve up images for processing and display wherein the dental image acquisition software interfaces with other applications via either an SDK driver or a TWAIN driver whereby the dental image acquisition software can perform preprocessing on the image before transferring to an imaging application, including but not limited noise filtering, sharpening, contrast enhancement or histogram equalization. The driver processes the encrypted license code stored in a sensor memory and determines if license code is expired based on the computer clock, exposure counter or other desired limits so that the intraoral sensor periodically checks an online server for an updated encrypted license code and stores new license code in the sensor memory. The driver also controls operation of the intraoral sensor on a computer and uses software which operates on the computer not only in real time, but also in the background so that the control center and licensing management system controls image acquisition and settings for the intraoral sensor so that it can be used with almost any dental imaging software on the market. An updated license code is created and stored on the server when a new periodic subscription payment is made. The intraoral sensor periodically checks and downloads any updated license code so that failure to make a payment will result in no license code update being created and eventual expiration of the old license code. The sensor licensing system requires periodic connection of the intraoral sensor to the internet for license checks. The encrypted licensing code incorporates date and image counter checking to prevent tampering and includes a mechanism which detects potential expiration of sensor license and notifies user of need to either update license or connect sensor to a server to check/update licensing status. The control center for an intraoral sensor monitors the performance of the intraoral sensor during use and notifies the user of problems.

Still further referring to FIG. 7 the dose optimization tool guides user to perform initial exposure measurements of an x-ray head for optimal signal level to minimize patient exposure to ionized radiation thereby ensuring optimal image quality while ensuring that the lowest possible x-ray generator settings to achieve diagnosable x-ray images. The dose optimization tool recommends ideal dose/time settings for use based on adult/child and different x-ray energies and includes a real-time signal monitor which monitors image level and will continue to monitor exposure of every image and notify the operator if signal is too high or low immediately after images acquisition during clinical use. The real-time monitor operates in the background thereby allowing it to be used independent of the imaging application software being used. The real-time monitor counts sensor usage and records data such as total exposures. The control center and licensing management system not only saves and stores recent images to allow for remote review and diagnosis of sensor issues during debugging, but also provides integrated support which is live with built-in support tools operating in the background, independent of the imaging application and monitors real time for software or sensor firmware updates.

Referring to FIG. 7 in conjunction with FIG. 2 the control center and licensing management system 520 manages licenses, licensing data, tamper protection, x-ray dose optimization, exposure monitoring and other information for use with the intraoral sensor 120. The control center and licensing management system for dental imaging operates in both real time and the background to control image acquisition and settings for the intraoral sensor 120 so that it can be used with almost any imaging software on the market. The control center and licensing management system 520 for dental imaging also monitors the sensor performance during use and notifies the user of problems. Image acquisition software acts as the primary driver for the intraoral sensor 120 and interfaces with other imaging applications to acquire and serve up images for processing and display. The interface with other applications can either be direct using a software development kit (“SDK”) or using a TWAIN data source. The software can perform pre-processing on the image before transferring to the imaging application, including noise filtering, sharpening, histogram equalization.

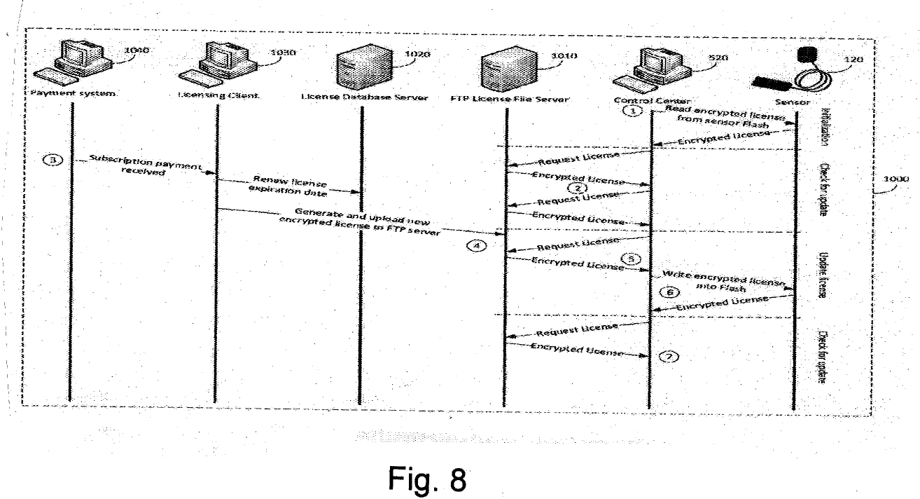

Referring to FIG. 8 in conjunction with FIG. 2 and FIG. 3 the control center and licensing management system 520 includes at least one intraoral sensor 120, at least one control center 520, an FTP license server 1010, a license database server 1020 with a memory 1021 containing licenses, a licensing client 1030 and a payment system 1040. These are the involved parties required to handle the x-ray sensor license distribution and enforcement.

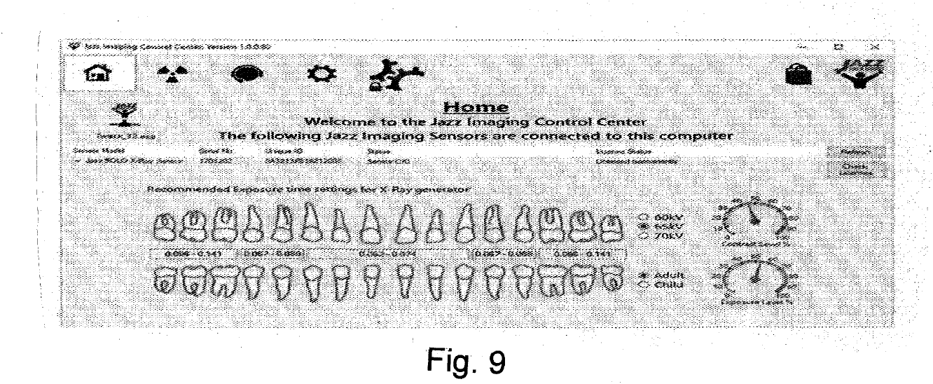

Referring to FIG. 9 in conjunction with FIG. 8 the interaction of at least one intraoral sensor 120, at least one control center 520, the FTP license server 1010, the license database server 1020 with a memory 1021 containing licenses, the licensing client 1030 and the payment system 1040 is as described below. The FTP license file server 1010 has a processor and license file storage coupled to the processor. When the intraoral sensor 120 is connected to the computer, the control center and licensing management system 520 reads the encrypted license from the flash memory. The intraoral sensor 120 is switched ON or OFF depending on license contents. When the control center and licensing management system 520 reads the license from the intraoral sensor 120, the control center and licensing management system 520 periodically requests encrypted licenses from the FTP license file server 1010 and determines whether it has been updated compared to the license read from the intraoral sensor 120. The payment module 1040 is sent a scheduled payment. When the scheduled payment is received, the license will be extended. The FTP license file server 1010 receives a new encrypted license. The control center and licensing management system 520 requests and receives the updated encrypted license file and determines that the encrypted license file has changed. The new encrypted license is stored inside the flash memory inside the intraoral sensor 120. The control center and licensing management system 520 periodically reads the encrypted license file from the FTP license file server 1010 and determines whether it has been updated compared to the license read from the intraoral sensor 120.

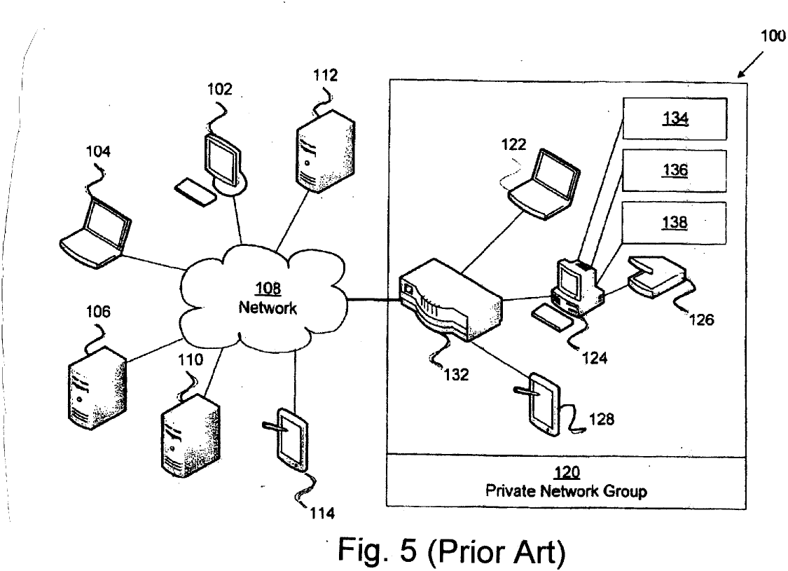

Referring still to FIG. 5 in conjunction with FIG. 4 the sensor licensing system 1000 of the control center and licensing management system 520 provides live license control of the intraoral sensor 120 with no user interaction by contacting a server for an encrypted licensing code which incorporates date and image counter checking to prevent tampering. This allows the intraoral sensor 120 to be turned on/off remotely if a periodic subscription payment is not made. This requires that license checks be made requiring periodic connection of the intraoral sensor and computer to the internet. The sensor licensing system 1000 includes an on/off mechanism, a driver, and a sensor. The on/off mechanism is coupled to the intraoral sensor and has an encrypted licensing code so that the on/off mechanism turns on and off the intraoral sensor. The driver processes the encrypted license code stored in the sensor memory and determines if license code is expired based on the computer clock, exposure counter or other desired limits. The sensor periodically checks an online server for an updated encrypted license code and stores new license code in sensor memory. The single control may be located on a plurality of individual computers and can handle licensing of multiple intraoral sensors 120. The updated license code is created and stored on the server when a new periodic subscription payment is made. The sensor periodically checks and downloads any updated license code. Failure to make a payment will result in no encrypted license code update being created and eventual expiration of the old, encrypted license code. License updates are created upon receiving subscription renewal payments and the transfer and storage of the updated license files involves encryption of the license information to prevent tampering with the license file contents. Since the x-ray sensor subscription plans typically involves setting and maintaining an expiration date for the license then it is of utmost importance that the licensing supervision system in the control center continuously verifies that the “system time” is correct and not being tampered with. This involves checking the PC’s real time clock against an external clock source (network time via NTP) as well as utilizing the x-ray sensors non-volatile memory to keep track of the last time the sensor was used. The primary concern is to make sure no one adjusts the time backwards to extend the valid license period so the basis for the tamper protection is to verify that the system time is always moving forward and never jumps back.

Referring still further to FIG. 9 in conjunction with FIG. 8 the control center and licensing management system 520 includes a driver, software including dental image acquisition software, a monitor, and a dose optimization tool. The driver controls the operation of the intraoral sensor on a computer. The software of the control center and licensing management system operates on the computer not only in real time, but also in the background for the control center and licensing management system 520 to control image acquisition and settings for the intraoral sensor so that it can be used with almost any dental imaging software on the market. The software of the control center and licensing management system 520 acts as the primary driver for the intraoral sensor and interfaces with other imaging applications to acquire and serve up images for processing and display. The control center software interfaces with other applications via either an SDK driver or a TWAIN driver. The software of the control center and licensing management system 520 can perform preprocessing on the image before transferring to an imaging application, including but not limited to noise filtering, sharpening, contrast enhancement or histogram equalization. A monitor checks the performance of the intraoral sensor during use and notifies the user of problems. The dose optimization tool guides user to perform initial exposure measurements of an x-ray head for optimal signal level to minimize patient exposure to ionized radiation thereby ensuring optimal image quality while ensuring that the lowest possible x-ray generator settings to achieve diagnosable x-ray images. The control center 520 saves and stores recent images to allow for remote review and diagnosis of sensor issues during debugging. The control center provides integrated support which is live with built-in support tools operating in the background, independent of the imaging application. The software of the control 520 monitors real time for software or sensor firmware updates.

The control center and licensing management system 520 involves the handling of license distribution and license enforcement for intraoral x-ray sensors so that it is kept generic to the various integrations of x-ray sensor products into 3rd party imaging software applications. The license distribution and enforcement are centered round a “control center” application that runs as a background service. The primary function of the control center and licensing management system 520 is to enforce and renew the licenses and to serve as a single point of integration for third party applications. Since the control center and licensing management system 520 is always required then it can perform background tasks such as image pre-processing and supervision of the image exposure level without any manual intervention by the end-users. The control center and licensing management system 520 serves as a convenient tool to be used for troubleshooting any issues that are reported from the field.

Referring again to FIG. 8, the dose calibration tool of the control center and licensing management system 520 guides user to perform initial measurement of each individual x-ray heads for optimal dose without exposing the patient to ionized radiation. The dose optimization tool also ensures an optimal image quality while ensuring that the lowest possible x-ray generator settings are set, avoiding overdosing of patients. The dose optimization tool further recommends ideal dose/time settings for use based on Adult/Child selections for different anatomic regions (like anterior, posterior) since the density of the bones and teeth varies accordingly. A real time dose monitor, after dose optimization, will continue to monitor exposure of every image and notify the operator if dose is too high or low immediately after images acquisition during clinical use. This is currently done by competitive imaging applications, but the subject dose optimization tool operates in the background thereby allowing it to be used independent of the imaging application being used. An exposure monitor counts sensor usage and records data such as total exposures and dose. A recent image storage system saves recent images to allow for remote review and diagnosis of sensor issues during debugging. An integrated support system includes live, built-in support tools operating in the background, independent of the imaging application. The intraoral sensor receives updates and monitors real time for software or sensor firmware updates.

Referring to FIG. 9 in conjunction with FIG. 7 and FIG. 8 the control center and licensing management system 520 for an intraoral sensor includes an on/off mechanism which is coupled to the intraoral sensor and a driver which is coupled to the on/off mechanism. The on/off mechanism has an encrypted licensing code so that the on/off mechanism turns on and off the intraoral sensor. The driver processes the encrypted license code stored in a sensor memory and determines if license code is expired based on the computer clock, exposure counter or other desired limits so that the intraoral sensor periodically checks an online server for an updated encrypted license code and stores new license code in the sensor memory. The control center and licensing management system monitors performance of the intraoral sensor during use and notifies the user of problems. The driver controls operation of the intraoral sensor on a computer and uses software which operates on the computer not only in real time, but also in the background so that the control center and licensing management system controls image acquisition and settings for the intraoral sensor so that it can be used with almost any dental imaging software on the market. An updated license code is created and stored on the server when a new periodic subscription payment is made so that the intraoral sensor periodically checks and downloads any updated license code so that failure to make a payment will result in no license code update being created and eventual expiration of the old license code.

The on/off mechanism has an encrypted licensing code and whereby the on/off mechanism turns on and off the intraoral sensor and wherein the driver processes the encrypted license code stored in a sensor memory and determines if license code is expired based on the computer clock, exposure counter or other desired limits whereby the intraoral sensor periodically checks an online server for an updated encrypted license code and stores new license code in the sensor memory and whereby the control center and licensing management system monitors performance of the intraoral sensor during use and notifies the user of problems. The on/off mechanism also turns on and off the intraoral sensor so that the driver processes the encrypted license code stored in a sensor memory and determines if license code is expired based on the computer clock, exposure counter or other desired limits whereby the intraoral sensor periodically checks an online server for an updated encrypted license code and stores new license code in the sensor memory and whereby the control center and licensing management system provides integrated support which is live with built-in support tools operating in the background, independent of the imaging application

The control center and licensing management system also includes a dose optimization tool coupled to the intraoral sensor and a real-time monitor. The dose optimization tool guides user to perform initial exposure measurements of an x-ray head for optimal signal level to minimize patient exposure to ionized radiation thereby ensuring optimal image quality while ensuring that the lowest possible x-ray generator settings to achieve diagnosable x-ray images. The dose optimization tool recommends ideal dose/time settings for use based on adult/child and different x-ray energies. The dose optimization tool includes a real-time signal monitor which monitors image level and will continue to monitor exposure of every image and notify the operator if signal is too high or low immediately after images acquisition during clinical use. The real-time monitor operates in the background thereby allowing it to be used independent of the imaging application software being used so that the real-time monitor counts sensor usage and records data such as total exposures. The control center and licensing management system saves and stores recent images to allow for remote review and diagnosis of sensor issues during debugging so that the control center and licensing management system provides integrated support which is live with built-in support tools operating in the background, independent of the imaging application. The control center and licensing management system also monitors real time for software or sensor firmware updates.

The control center and licensing management system requires periodic connection of the intraoral sensor to the internet for license checks. The encrypted licensing code incorporates date and image counter checking to prevent tampering.

The control center and licensing management system includes a mechanism which detects potential expiration of sensor license and which notifies user of need to either update license or connect sensor to a server to check/update licensing status so that dental image acquisition software acts as the primary driver for the intraoral sensor and interfaces with other imaging applications to acquire and serve up images for processing and display. The dental image acquisition software interfaces with other applications via either an SDK driver or a TWAIN driver so that the dental image acquisition software can perform preprocessing on the image before transferring to an imaging application, including but not limited noise filtering, sharpening, contrast enhancement or histogram equalization.

From the foregoing, it is seen that a control center and licensing management system which manages licenses, licensing data, tamper protection, x-ray dose optimization, exposure monitoring, and other information has been described.

Accordingly, it is intended that the foregoing disclosure and no showing made in the drawing should be considered only as an illustration of the principle of the present invention.; and a driver coupled till result in no encrypted license code update being created and eventual expiration of the old, encrypted license code.

What is claimed is:

1. A control center and licensing management system for an intraoral sensor wherein said control center and licensing management system comprises: an on/off mechanism coupled to the intraoral sensor; and a driver coupled to said on/off mechanism wherein said on/off mechanism has an encrypted licensing code and whereby said on/off mechanism turns on and off the intraoral sensor and wherein said driver processes said encrypted license code stored in a sensor memory and determines if license code is expired based on the computer clock, exposure counter or other desired limits whereby the intraoral sensor periodically checks an online server for an updated encrypted license code and stores new license code in said sensor memory.

2. A control center and licensing management system for an intraoral sensor according to claim 1 wherein said control center and licensing management system monitors performance of the intraoral sensor during use and notifies the user of problems.

3. A control center and licensing management system for an intraoral sensor according to claim 1 wherein said driver controls operation of the intraoral sensor on a computer and uses software which operates on said computer not only in real time, but also in the background so that said control center and licensing management system controls image acquisition and settings for the intraoral sensor so that it can be used with almost any dental imaging software on the market.

4. A control center and licensing management system for an intraoral sensor according to claim 1 wherein an updated license code is created and stored on the server when a new periodic subscription payment is made and wherein the intraoral sensor periodically checks and downloads any updated license code so that failure to make a payment will result in no license code update being created and eventual expiration of the old license code.

5. A control center and licensing management system for an intraoral sensor according to claim 1 wherein said sensor licensing system includes a dose optimization tool coupled to the intraoral sensor.

6. A control center and licensing management system for an intraoral sensor according to claim 5 wherein said dose optimization tool guides user to perform initial exposure measurements of an x-ray head for optimal signal level to minimize patient exposure to ionized radiation thereby ensuring optimal image quality while ensuring that the lowest possible x-ray generator settings to achieve diagnosable x-ray images.

7. A control center and licensing management system for an intraoral sensor according to claim 1 said control center and licensing management system requires periodic connection of the intraoral sensor to the internet for license checks.

8. A control center and licensing management system for an intraoral sensor according to claim 1 wherein said encrypted licensing code incorporates date and image counter checking to prevent tampering.

9. A control center and licensing management system for an intraoral sensor according to claim 8 wherein said control center and licensing management system includes a mechanism which detects potential expiration of sensor license and which notifies user of need to either update license or connect sensor to a server to check/update licensing status.

10. A control center and licensing management system for an intraoral sensor according to claim 8 wherein dental image acquisition software acts as the primary driver for the intraoral sensor and interfaces with other imaging applications to acquire and serve up images for processing and display wherein said dental image acquisition software interfaces with other applications via either an SDK driver or a TWAIN driver whereby said dental image acquisition software can perform preprocessing on the image before transferring to an imaging application, including but not limited noise filtering, sharpening, contrast enhancement or histogram equalization.

11. A control center and licensing management system for an intraoral sensor according to claim 6 wherein said dose optimization tool recommends ideal dose/time settings for use based on adult/child and different x-ray energies.

12. A control center and licensing management system for an intraoral sensor according to claim 11 wherein said dose optimization tool includes a real-time signal monitor which monitors image level and will continue to monitor exposure of every image and notify the operator if signal is too high or low immediately after images acquisition during clinical use.

13. A control center and licensing management system for an intraoral sensor according to claim 12 wherein said real-time monitor operates in the background thereby allowing it to be used independent of the imaging application software being used.

14. A control center and licensing management system for an intraoral sensor according to claim 12 wherein said real-time monitor counts sensor usage and records data such as total exposures.

15. A control center and licensing management system for an intraoral sensor according to claim 3 wherein said control center and licensing management system saves and stores recent images to allow for remote review and diagnosis of sensor issues during debugging.

16. A control center and licensing management system for an intraoral sensor according to claim 15 wherein said control center and licensing management system provides integrated support which is live with built-in support tools operating in the background, independent of the imaging application.

17. A control center and licensing management system for an intraoral sensor according to claim 15 wherein said control center and licensing management system monitors real time for software or sensor firmware updates.

18. A control center and licensing management system for an intraoral sensor wherein said control center and licensing management system comprises: an on/off mechanism coupled to the intraoral sensor; and a driver coupled to said on/off mechanism wherein said on/off mechanism has an encrypted licensing code and whereby said on/off mechanism turns on and off the intraoral sensor and wherein said driver processes said encrypted license code stored in a sensor memory and determines if license code is expired based on the computer clock, exposure counter or other desired limits whereby the intraoral sensor periodically checks an online server for an updated encrypted license code and stores new license code in said sensor memory and whereby said control center and licensing management system monitors performance of the intraoral sensor during use and notifies the user of problems.

19. A control center and licensing management system for an intraoral sensor wherein said control center and licensing management system comprises: an on/off mechanism coupled to the intraoral sensor; and a driver coupled to said on/off mechanism wherein said on/off mechanism has an encrypted licensing code and whereby said on/off mechanism turns on and off the intraoral sensor and wherein said driver processes said encrypted license code stored in a sensor memory and determines if license code is expired based on the computer clock, exposure counter or other desired limits whereby the intraoral sensor periodically checks an online server for an updated encrypted license code and stores new license code in said sensor memory and whereby said control center and licensing management system provides integrated support which is live with built-in support tools operating in the background, independent of the imaging application.

20. A control center and licensing management system for an intraoral sensor according to claim 19 whereby said control center and licensing management system monitors performance of the intraoral sensor during use and notifies the user of problems.
